(19) United States
(12) Patent Application Publication
(10) Pub. No.: US 2004/0063624 A1
(43) Pub. Date: Apr. 1, 2004

(54) Method of inhibiting the transition of free HIV virus through the cllular mucosal barrier

(76) Inventors: Andreas Kage, Berlin (DE); Eskandar Shoolian, Berlin (DE); Georg Pauli, Berlin (DE)

Correspondence Address: McGLEW AND TUTTLE, John James McGlew, Scarborough Station, Scarborough, NY, 10510-0827, US

(21) Appl. No.: 10/669,767

(22) Filed: Sep. 24, 2003

Related U.S. Application Data
(63) Continuation of application No. 09/540,089, filed on Mar. 31, 2000, now abandoned.
(60) Provisional application No. 60/128,304, filed on Apr. 8, 1999.

(51) Int. Cl. A61K 38/16; A61K 31/715

(52) U.S. Cl. 514/8; 514/54

(57) ABSTRACT

The invention provides a method of inhibiting the transition of free HIV virus carrying an envelope glycoprotein gp120/gp160 through the cellular mucosal barrier of an organism, wherein said glycoprotein is blocked by increasing in the region of said mucosal barrier the concentration of compounds comprising an oligomannosyl glycan residue and/or compounds comprising mimic molecules of an oligomannosyl glycan, wherein the link of said glycoprotein to said HIV virus remains essentially unaffected. The invention further provides useful pharmaceutical compositions and methodes for preventing an HIV infection and/or treating an HIV infection.

### FIELD OF THE INVENTION

The invention relates to the epithelial uptake and transport of cell-free HIV virus, in particular HIV type 1. The invention further relates to methods suitable for inhibiting the epithelial uptake and transport of HIV virus, to methods for preventing an HIV infection of an organism and treatment of organisms infected with HIV. The invention furthermore relates to pharmaceutical compositions useful for preventing HIV infection or treating organism infected with HIV.

### BACKGROUND OF THE INVENTION

Little is known about the mechanisms of Human immunodeficiency virus (HIV) virus entrance into an organism through the cellular mucosal barrier. HIV must pass epithelial cells which are part of the mucosal barrier to infect CD4+ cells (Frankel, S. S. et al., 1996, Science 272, 115-117; Ludewig, B. J. et al., 1995, J. Gen. Virol. 76, 1317-1325). Virus entry may occur if the integrety of the mucosa is compromised. Alternatively, entry via receptor-mediated uptake that involves receptors distinct from CD4, which is not expressed on epithelial cells may be feasable. Virus transport through the epithelial cell monolayers is suggested by several experiments. During incubation of HIV type 1 infected mononuclear blood cells, with no cell-free virus present, on the apical site of monolayers of immortalized cells a basolateral release of infectious virus was shown (Bomsel, M., 1997, Nat. Med. 3, 42-47). Furthermore, infection of neonate and adult macaques with cell-free simian immunodeficiency virus via the upper alimentary tract has been demonstrated, suggesting virus transport through the mucosal barrier (Baba, T. W. et al., 1995, Science 267, 1820-1825; Baba, T. W. et al., 1996, Science 268, 2395-2398). However, no information on the penetration of HIV through primary human epithelial cells has been made available prior to the inventors findings.

The inventors have published a scientific article on the subject matter of the invention in the Journal of Virology, 1998, 4231-4236 (exact publication date: Apr. 10, 1998). This article is incorporated into the present application by way of reference.

### OBJECT OF THE INVENTION

It is an object of the invention to elucidate the epithelial uptake and transport of cell-free human HIV. A further object is to provide a method for inhibiting the transition of free HIV through the cellular mucosal barrier, in particular through a layer of epithelial cells. A further object is to provide a method for preventing HIV infection of an organism by preventing said transition. A further object is to provide a method for preventing reinfection of an infected organism under HIV treatment. A further object is to provide compounds useful for pharmaceutical compositions for said medical methods.

### BRIEF SUMMARY OF THE INVENTION

The invention provides a method of inhibiting the transition of free HIV virus carrying an envelope glycoprotein gp120/gp160 through the cellular mucosal barrier of an organism, characterized in that said glycoprotein is blocked by increasing in the region of said mucosal barrier the concentration of a compound comprising an oligomannosyl glycan residue and/or of a compound comprising, preferably being, a mimic molecule of an oligomannosyl glycan residue, wherein the link of said glycoprotein to said HIV virus remains essentially unaffected.

It is essential for the invention that the said compound does not strip the glycoprotein from the virus. Instead, the compound attaches to specific binding sites of the glycoprotein and inhibits the attachment of the glycoprotein to corresponding receptors located at the cell surface. In consequence, the blocked virus does not any more possess the capability to enter and transit epithelial cell layers, with the result that the organism is protected from HIV infection in course of sexual contacts and the like.

Mimic molecules are structures which act biologically like the compound they mimic despite the different chemical structure. In most cases the affinity imparting part of the mimic molecule has a three dimensional structure highly similar to the “original” molecule in a “lock-key” sense, the mimic molecule being the “key” to the binding site of gp120/gp160. Particularily promising mimic molecules are the so called aptamers, which are nuclein acids, in particular RNAs. The group of mimic molecules also comprises aptamer analogues being constructed from amino acids, i.e. peptides or proteins. In any case the “lock-key” konfiauration is essential.

### DETAILED DESCRIPTION OF THE INVENTION

In an preferred embodiment of the invention the increase of the concentration of said compound in said barrier is effected by local administration of said compound to said barrier, in particular epithelial cell barrier. The administration may e.g. be performed by manual application of a pharmaceutical composition according to the invention to the vaginal region of a women prior to a sexual contact. Another form of administration may be to apply the pharmaceutical compound to an inner and/or outer surface of a mechanical contraceptive like a cap, diaphragm, or condome prior to a sexual contact. The term local administration does not only relate to a direct application of the pharmaceutical composition to epithelial tissue but also to an application to the space adjacent to the epithelial tissue. It is e.g. also possible to administer the pharmaceutical composition in form of a foam or the like. In any case it is of importance to adiminster the pharmaceutical composition at least to body parts or hollow body parts, which may be exposed to HIV virus contaminated material.

Alternatively, the concentration of said compound in said barrier may be effected by stimulation of the β-andrenergic system within the said organism. Such stimulation may e.g. be achieved under physical stress. As a further alternative the increase of the concentration of said compound in said barrier may be effected by inhibition of the endogenic processing of glycans, e.g. by administration of an inhibitor selcted from the group consisting of “desoxymannojirimycin, swainsonine, desoxynojirimycin, and mixtures thereof”. As a result, the N-glycan-processing in the organism, in particular with respect to high mannose glycanes and/or hybride glycanes, is reduced or interrupted, so that oligomannosyl residues occuring naturally as intermediate product(s) are not further processed and the concentration of the naturally in vivo formed oligomannosyl residues is enhanced.

It is preferred that the said compound is selected from the group consisting of “N-glycans, mannan, high-mannose type glycans, hybrid-type glycans, complex-type glycans, α-methylmannopyranoside, mucine, yeasts, beer yeasts, extracts of Aloe Vera, and mixtures and/or derivatives thereof”, wherein the mannose residues of said compound are essentially non-sulphated. The mannose residues involved in blocking the said glycoprotein are non-ionic. This does, however, not exclude the presence of e.g. sulfated or otherwise ionic residues in groups of the said compound which are not involved in blocking the said glycoprotein. An example for such a compound is mucine. The essential part of said compound is formed by a plurality of mannose molecules linked via glycosidic bonds. The so-formed glycans may be linear or branched, e.g. comprising five mannose groups branched at a “base” mannose group. The glycans may comprise one or two N-acetylglucoseamine groups, in case of a branched glycan at the “root” of the “tree”. High mannose type glycans carry further mannose groups in addition to the said five mannose groups and may be derived from RNAse B. Hybrid type glycans may comprise branches of mannose molecules, from one of which a part of the chain has been cleaved off. Hybrid type glycans may e.g. be derived from ovalbumin. Complex type glycans typically (but not necessarily) comprise three mannose molecules in branched configuration, wherein the braches are extended with sugar units other than mannose. Complex type glycans may be derived from fetuin.

The invention is further directed to a pharmaceutical composition capable of inhibiting the transition of free HIV virus carrying an envelope glycoprotein gp120/gp160 through the mucosal barrier of an organism, which contains at least one compound comprising an oligomannosyl glycan residue and/or at least one compound comprising, preferably being, a mimic molecule of an oligomannosyl glycan residue for blocking said glycoprotein, wherein the link of the said glycoprotein to said HIV virus remains essentially unaffected and to a pharmaceutical composition capable of inhibiting the transition of free HIV virus carrying an envelope glycoprotein gp120/160 through the mucosal barrier of an organism, which contains at least one inhibitor of the endogenic processing of glycans. In the latter case the pharmaceutical composition may comprise an inhibitor selected from the group consisting of “desoxymannojirimycin, swainsonine, desoxynojirimycin, and mixtures thereof”. In general, the details provided above as to the method of inhibition apply in a corresponding manner to the pharmaceutical composition of the invention.

In case of an organist being infected the invention may be used to prevent reinfection during a medical treatment procedure directed to removing the virus from infected compartments. In this case a combination of the pharmaceutical composition described above with a pyrimidine nucleoside analogue capable of inhibiting reverse transcriptase is of advantange. The compound of the invention may be combined with the nucleoside analogue in one single composition component although it may be advisable to formulate the compound of the invention in a package component separate from a package component containing the nucleoside analogue. This is in particular true in case of the compound of the invention comprising an oligomannosyl glycan residue. In a preferred embodiment of the invention the nucleoside analogue is 3′-Azido-3′-desoxythymidin. This compound is also known as Zidovudin or AZT. Additionally, further nucleoside analogues like 3TC and/or a protease inhibitor may be used like in the known HAART therapy.

The invention is furthermore directed to a method for preventing an infection of an organism with free HIV virus carrying an envelope glycoprotein gp120/gp160 wherein a compound comprising an oligomannosyl glycan residue and/or a compound comprising, preferably being a mimic molecule of an oligomannosyl glycan residue is administered locally to epithelial tissues of said organism, wherein said glycoprotein is blocked by said compound and wherein the link of said glycoprotein to said HIV virus remains essentially unaffected, to a method for preventing an infection of an organism with free HIV virus carrying an envelope glycoprotein gp120/gp160, wherein an increase of the concentration of a compound comprising an oligomannosyl glycan residue in said barrier is effected by stimulation of the β-andrenergic system within the said organism, to a method for preventing an infection of an organism with free HIV virus carrying an envelope glycoprotein gp120/gp160, wherein an increase of the concentration of a compound comprising an oligomannosyl glycan residue in said barrier is effected by inhibition of the endogenic processing of glycans, and to a method for treating an organism infected with HIV virus carrying an envelope glycoprotein gp120/gp160 wherein a compound comprising an oligomannosyl glycan residue and/or a compound comprising, preferably being a mimic molecule of a oligomannosyl glycan residue is administered to said organism, wherein said glycoprotein is blocked by said compound and wherein the link of said glycoprotein to said HIV virus remains essentially unaffected, and wherein a pyrimidine nucleoside analogue capable of inhibiting reverse transcriptase is administered to said organism, wherein the administration of said compound and of said nucleoside analogue is performed simultaneously or sequentially, e.g. alternating. The details outlined above in context with the other aspects of the invention apply in an analogue way. In any case, the compound may consist of an oligomannosyl glycan residue.

In the following the invention is explained by way of non-limiting examples in more detail. The figures show:

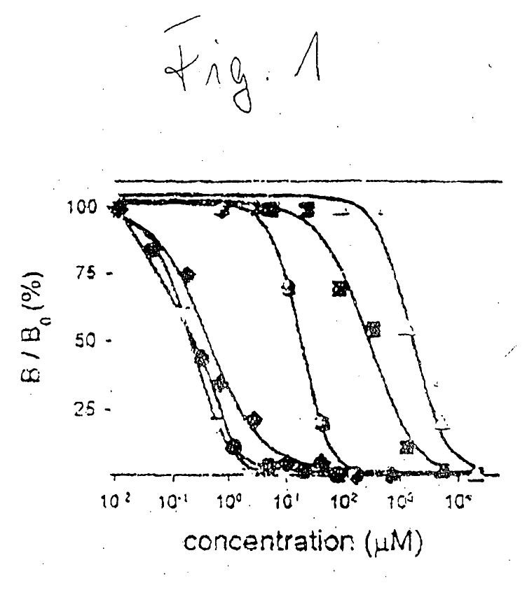

FIG. 1: Binding of biotinyl-mannan to immobilized gp120.

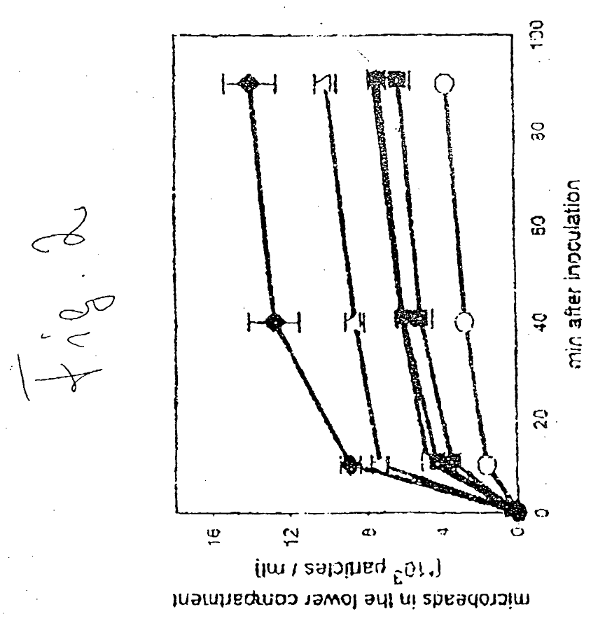

FIG. 2: Particles in the basal chamber of a test apparatus after 10, 40 and 90 min. of incubation at 37° C.

In the experiments presented the following methods and materials were used.

Primary Culture of Epithelial Cells.

Epithelial cells were obtained from biopsies of the gingiva of a healthy male donor. The biopsies were washed several times with phosphate-buffered saline and cultured after trypsinization in Dulbecco modified Eagle medium containing 10% fetal calf serum (FCS). Fibroblast growth was suppressed by the addition of recombinant epidermal growth factor (10 μg/liters Sigma, Deisenhofen, Germany) to the culture medium. The epithelial character of the primary cells and the formation of tight junctions were confirmed morphologically by electron microscopy.

The expression of cytokeratin and CD4 receptor was investigated by immunocytology. Cells cultivated on coverslips for 5 days were washed with cold phosphate buffered saline and fixed with acetone for 10 min at room temperature. The nonspecific binding sites were blocked with 50 mM Tris-50 mM NaCl-10 mM CaCl₂-0,1% normal goat serum pH 7,5. After being washed the cells were incubated with anticytokeratin ceceptot (CK5:ICN ImmunoBiological) or anti CD4 receptor (OKT4: Dianova) and the primary antibodies were visualized by alkaline phosphatase and monoclonal antialkaline phosphatase staining (Cordell J. L. et al., 1984, J. Histochem. Cytochem., 32, 219.229).

Two-Compartment Culture System.

For transepithelial transport experiments, primary epitheilal cells (10⁴/ml) were grown on a polycarbonate filter membrane (9 mm diameter 3,0 μm pore diameter, Becon Dickinson) separating a basal and an apical chamber and cultivated for 10 days until confluence was observed. The development of an epithelial monolayer was examined by confocal laser microscopy. To further test for confluence a fluorescein solution (0,2 mg/ml) or fluorescent paricles (10⁶ particles/ml each particle 0,1 μm in diameter) were added to the apical chamber and fluorescence activity in the basal chambers was measured after 45 min of incubation at 4° C. to inhibit cell membrane diffusion. For calibration of paracellular diffusion membranes were covered with a layer of 15% polyacrylamide gel leaving free circular areas by placing small cylinders of defined size on the filter before gel casting. Fluorescence activity which diffused from the apical to the basal chamber through different areas of uncovered epithelial cells was measured after the 45 min. incubation.

Viral Transepithelial Transport.

HIV-1 strain IIIb (1,8×10⁴ 50% tissue culture infective doses [TCID₅₀/ml]) harvested from HIV-1 infected H9 cells was cleared from all debris by centrifugation (10 min. 200×g) and filtered through a 0,2 μm pore size filter membrane. The cell-free virus was diluted 1:10 with Hanks buffer and was placeed in the apical compartment (for details see below). After a 45 min incubation, medium from the lower compartment was harvested. The amount of infectious HIV-1 in the basal chamber of the culture unit was determined by a standard tritation assay Tritation of HIV was performed in triplicate in 24-well tissue culture plates on MT 4 cells seeded at a concentration of 2×10⁴/ml. Samples were diluted serially (1:10) in culture medium (RPMI 1640 supplemented with 10% FCS and 5% glutamine). The titration was evaluated between 10 and 14 days postinfection when a prominent cytopathic effect (CPE) was visible. Medium was replaced twice a week with cells being diluted as required. Values of TCID₅₀ per milliliter were determined as described in Reed C. J. et al., 1938, Am. J. Hyg., 27, 494.497. All experiments were performed in triplicate.

Inhibition Studies.

For inhibition studies on MT4 and epithelial cells mannan (5 mg/ml final concentration) α-methyl-mannopyramoside (aMMP; 100 mM final concentration ) or mucin (30 mg/ml) was added to the dilution buffer. Monosaccharide analysis after hydrolysis of the mucin showed that the mannose content was about 1% of the total mass. The HIV-1 specificity of the CPE in MT4 cells was confirmed by determination of p24 core protein content by a p24 antigen capture assay (Coulter). Control experiments indicated that relevant concentrations of the glycoconjugate inhibitors neither reduced the titer of HIV-1 nor inhibited the CPE in MT4 cells. Experiments were done in triplicate.

Viral Intake and Release.

HIV-1 strain IIIb (1,8×10⁵ TCID₅₀/ml) was placed on epithelial cells (10⁶/petri dish). For inhibition studies mannan (5 mg/ml final concentration) aMMP (100 mM) or mucin (30 mg/ml) was added to the culture medium. After a 1-h incubation, the cells were washed three times with trysin (0,25%) and incubated for 10 min at 37° C. with trypsin solution to inactivate all virus particles adsorbed at the cell surface. The cells were harvested and subcultured for different incubation times (see. Table 1). Subsequently, the cell-free supermatant of each subculture was titrated on MT4 cells as described above.

Preparation of Biotinyl-Mannan.

To avoid non-mannosyl-mediated binding of mannan, the oligopeptide tail of mannan was digested by proteinase K treatment (20 μg of protease K/100 mg of mannan) for 2 h at 37° C., resulting in a protein content reduction of from 5% to below 0,1% of the total mass Mannan was separated from free amino acids as well as from the enzyme molecules by affinity chromatography with Galanthus nivalis agglutinin (GNA). Bound mannan was specifically eluted with 100 mM aMMP. The residual peptide core of mannan (80 mg of mannan/ml of Na₂CO₃; 50 mM; pH 8,5) was biotinylated by overnight incubation with HN-hydroxy-succinimide-capronyl-biotin (0,2 mg/ml). Biotinylated and non-biotinylated mannan molecules were separated from hydrolyzed capronyl-biotinyl by GNA affinity chromatography. In order to separate the biotinylated mannan conjugates from nonbiotinylated conjugates from mannan and a MMP the lipophilic biotinyl carbohydrates, i.e. mannan and a MMP, the lipophilic biotinyl conjugates were retained on a reversed-phase cartridge (SEP-PAC-Cartrigde, C₁₅: Waters, Eschborn, Germany) and eluted by a stepwise gradient of methanol-water (0 to 10% [vol/vol]) . The eluate was lyophilized and stored at −20° C. until use.

gp120 Preparation, Characterization of Lecitin-Like Activity, and Coupling to Microbeads.

Cell-free supernatant of HIV-1 strain IIIB infected human H9 cells was treated with 0,5% Nonident P-40 and protease inhibitor (phenylmethylsulfonyl fluoride; 5 mM). Debris was eliminated by ultracentrifugation at 100.000×g for 2 h at 4° C. The viral envelope glycoprotein was purified by GNA affinity chromatography as described in Gilljam G., 1993, AIDS Res. Hum. Retroviruses, 9, 431-438, followed by immunoaffinity chromatography using human serum immunoglobulins with high anti-HIV-1 gp¹²⁰ titers (demonstrated by Western blot analysis). The purity and specificity of the gp120 was confirmed by sodium dodecyl sulfate-polyacrylamide gel electrophoresis (SDS-Page) in conjunction with silver staining and by immunoblotting with HIV-1 gp120 specific monoclonal antibody (clone RLI6.76I; Immunotech. Hamburg-Germany). The sensitivity of the staining was enhanced by a luminol-containing substrate as described in Thorpe S. J., 1986, J. Clin. Pathol., 39, 1165-1176.

To test the lectin properties of the native HIV-1 gp120 the envelope glycoprotein was electrophoresed on a polyacrylamide-SDS gel, blotted into a polystyrol surface and stained with the biotinyl-mannan conjugate (Tris buffer, 1% glycine, 0,2% Tween 20, 5 mM CaCl₂, pH 7,3), a mouse monoclonal antibiotin antibody (Boehringer GmbH, Mannheim, Germany), and a secondary peroxidase labelled antimouse antibody (Dako, Hamburg, Germany), followed by incubation with a chemiluminescence substrate as described above. To reduce nonspecific binding of the monoclonal antibiotin antibody to the immobilized gp120, carbohydrates of the antibody were oxidized with periodate. The periodate oxidation eliminated the mannosyl-specific lectin binding of the monoclonal antibody after dot blotting. The purified HIV-1 gp120 was adsorbed onto a polystyrrol surface. The lectin-like binding properties of the immobilized gp120 were determined by incubating with biotin-labelled mannan overnight at 37° C., and the amount of gp120-bound biotinyl-mannan was quantified with a biotin-specific monoclonal antibody. Different concentrations of various soluble carbohydrates (high-mannose-type glycans [5 to 9 mannose molecules per glycan] derived from RNAse B), hybrid-type glycans (derived from ovalbumin), complex-type glycans (derived from fetuin: Oxford Glycosystem, Oxford, United Kingdom), aMMP, and glucose were coincubated with the biotinylated mannan complex (for details see text related to FIG. 1). The 50% inhibitory concentrations (IC₅₀) were estimated by four-parameter logistic spline interpolation after equilibrium incubation (Huoton, J. C. et al., J. Immunolog., 135, 2464-2473).

For transepithelial transport studies gp120 was covalently coupled to monodispersed carboxylated fluorescent microparticles (0,1 μm in diameter; Polysciences, Warlington, Pa.) as described previously (Molday R. S., 1975, J. Cell Biol., 64, 75-88). The active groups of the control beads and the remaining gp120 coated beads were blocked with glycine. The attachment of gp120 to the fluoresent particles was quantified by measuring the binding of a monoclonal anti-HIV gp120 antibody to the beads.

Transepithelial Transport of Particles.

The gp120-coated particles were diluted (10⁵ partices/ml) in Hanks buffer and placed in the apical chamber after the epithelial cells were washed three times with Hanks buffer. The apical chamber was transferred into a new basal chamber, and Hanks buffer was changed after 10, 40, 90 min of incubation. The buffer harvested at the indicated time points was centrifuged (14.000×g for 15 min), the pellet was resuspended and the fluorescence activity was measured. A combination of forskolin (FSK 10 μM ) and 3-isobutyl-1-methyl-xantin (IBMX, 10 μm) was used to study the effect of cyclic AMP (cAMP) and epithelial transport. Preincubation of the epithelial cells for 2 h increased the intracellular cAMP level eightfold (cAMP immunoassay: Biomol, Plymouth Meeting, Pa.). Glycine-coated microparticles were used as the control for nonspecific paracellular flow under the experimental conditions: the fluorescence of the controls was subtracted from the fluorescence-activities found in the respective experiments with gp120-coated particles.

Inhibition Experiments.

Before addition to the apical chamber, the gp120-coated particles (10⁵ particles/ml) were preincubated for 10 min with mannan (5 mg/ml final concentration) or aMMP (100 mM final concentration). After incubation for 10, 40 and 90 min at 37° C., the solution in the basal chamber was changed and the fluorescence activity was measured as described above.

To increase the amount of high-mannose-type glycans on the epithelial cells, cells were preincubated for 2 h with 10 mM desoxymannojirimycin (Fuhrmann U. E., 1984, Nature, 307, 755-788). To control the increase of high-mannose-type glycan expression, the filter membrane was cut and placed in 300 μl of lysis buffer (0,2 M Tris-HCl, 2% SDS, 0,1% dithiothreitol). After centrifugation at 3.000×g for 5 min. the glycoproteins of the supernatant were separated by SDS-PAGE and the glycoproteins blotted onto nitrocellulose were incubated with a GNA-digoxigenin conjugate (0,1 μg/ml Boehringer GmbH ) for 1 h and stained with an anti-digoxigenin-peroxidase conjugate (0,1 μg/ml, Boehringer GmbH) Bound peroxidase was determined after incubation of the nitrocellulose with a chemiluminescent substrate and exposure to photon-sensitive film (Kodak X-AR) as described previously (Thorpe S. J., 1986, J. Clin. Pathol., 39, 1165-1176).

TABLE 1
Detection at cell-free HIV 1 taken up by
epithelial monolayer

HIV concn (TCID50/ml) in the supernatant
after treatm. with:

| Time after | no | Mannan | aMMP | Mucin |
|---|---|---|---|---|
| trypsin treatm. | inhibitor | (5 mM) | (100 mM) | (1 mM) |
| 60 min. | 10³ | 10⁰ | 10¹ | 10¹ |
| 120 min. | 10³ | 10¹ | 10¹ | 10¹ |
| 160 min | 10³ | 10¹ | 10¹ | 10¹ |
| 24 h | 10³ | 10¹ | 10¹ | 10¹ |

For the data in Table 1 HIV-1 strain IIIb (1,8×10⁴ TCID₅₀/ml) was placed on epithelial cells (10⁶/petri dish) with or without inhibitors. After a 1-h incubation the cells were treated with trypsin to inactivate all extracellular HIV particles. Cells were incubated with fresh medium and the supernatants were harvested at different time points. Infectious virus in the supernatant was titrated on MT4 cells (see Materials and Methods). All experiments were performed in triplicate. The differences between the infectious titers with and without inhibitors are highly significant for all incubation times (P<0.001 by the Mann Whitney U test).

Results

The degree of paracellular leakage of the epithelial cell-monolayer was tested by incubation with flourescein- or glycine-coated fluorescent microbeads. With an uncovered membrane (maximal flow rate) about 2% of the upper-compartment fluorescence activity was detected in the lower compartment. In all experiments the paracellular flow was always less than 4% (mean: 1,8%) of the maximal flow rate, i.e. less than 0 05% of the input particles.

After cell- free infectious HIV-1 was placed on the epithelial monolayer the quantity of infectious virus on the basal side of the epithelial monolayer was determined by titration of infectious virus. Approximately 5% (10³ TCID₅₀/ml) of the virus placed in the upper compartment was found in the basal chamber after a 45 min. incubation. Preincubation with mannan (5 mM) or aMMP (100 mM) reduced the amount of infectious HIV 1 in the basal compartment by 1 order of magnitude (i.e. to 10² TCID₅₀/ml). Mucin inhibited the transepithelial transport of cell-free HIV-1 to a similar extent (10² TCID₅₀/ml). The differences between the results in the absence and presence of inhibitors are highly significant (P<0.001 by the Mann Whitney U test). Supernatant of uninfected epithelial cells did not induce a CPE. Epithelial cells were incubated with cell- free HIV-1 for 30 min., the cell supernatant was removed, and the cell surfaces were treated with trypsin. Infectious virus particles were released for several hours into the basal chamber (Table 1). Cellular uptake and release were inhibited by 2 orders of magnitude after coincubation of the virus and the epithelial cells with mannosyl derivatives (Table 1). Mucin could also significantly inhibit the viral uptake and release of cell-free HIV-1 (Table 1). Incubation of mannosyl derivatives with the CD4- indicator cells and cell-free HIV did not show any inhibition of CPE. Supernatants of epithelial cells without treatment with HIV did not induce CPE in MT4 cells.

After the dot blotting of native HIV-1, gp120 was shown to bind to biotinylated mannan. This binding was effectively inhibited by glycans with a terminal oligomannosyl structure. The IC₅₀ of high-mannose-type glycans (IC₅₀=0,20 μM), glycans of the hybrid type (IC₅₀=0,37 μM), and mannan (IC₅₀=0,24 μM) were comparable (FIG. 1). The IC₅₀ was 40 μM for complex-type glycans which contain a trimannosyl core. Monosaccharides such as aMMP and glucose also inhibited binding, but only at much higher concentrations (IC₅₀=275 μM for aMMP, IC₅₀=1.460 μM for glucose).

To demonstrate that transepithelial transport was mediated by HIV-1 gp120 and not by receptors from the H9 cell line used to grow the virus, fluorescent polystyrrol microspheres coupled to purified gp120 were placed in the apical chamber. The amount of gp120-coupled particles was quantified in the medium at the basal chamber, and the number of glycine-coated particles (control) was subtracted (FIG. 2). Compared to transport through unstimulated cells, the transport of gp120 coated particles through the epithelial monolayer was increased by 50% upon preincubation with a combination of FSK and IBMX compounds (FIG. 2). To increase the number of glycan receptors on the epithelial cells, the cells were preincubated with desoxymannojirimycin, which inhibits the mannosidase I in the Golgi apparatus. This pretreatment resulted in a further augmentation of the cAMP-stimulated increase of particles in the basal compartment (FIG. 2). The competitive inhibitors mannan and aMMP reduced the transport of gp120-coated particles in FSK- and IBMX-treated cells to about 50% of the level for unstimulated cells (FIG. 2).

At the beginning of the incubation of gp120-coated particles with epithelial cells a rapid increase of particles in the lower compartment was observed (FIG. 2). At later times (40 min of incubation) we did non detect any significant increase of gp120-coated particles in the basal chamber despite the fact that a high concentration of such particles in the apical chamber was present. Further evidence of transepithelial transport is provided by electron microscopy studies showing gp120-coated particles in the endosomes of epithelial cells.

Lectin staining after SDS-PAGE and subsequent Western blotting of epithelial cell lysates showed several mannosylated glycoproteins which might be involved in gp120 binding.

The described experiments imply that HIV-1 can be taken up by the epithelial monolayer or epithelial tissue without losing infectivity. The virus breaks through this barrier and finally leads to infection of the organism with HIV. Furthermore, it is demonstrated that the compounds described concur with the uptake mechanism with the result that the relevant sites of gp120/160 are blocked by the compound. This is evident from the presented data since these indicate that the receptors for HIV-1 gp120 on primary human epithelial cells are oligomannosyl residues of cellular surface glycoproteins interacting with the lectin-like domain on the HIV-1 gp120 molecules. Accordingly, blocking of gp120 results in the blocked virus losing its capability to break through the epithelial barrier and to enter the organism. Thus, infection is prevented. Since at least some of the compounds of the present invention may be formed endogenically, the methods of the invention do not only comprise administration of these compounds but also methods to increase the endogenic formation thereof. This may be of particular importance in situations wherein the endogenic formation is naturally reduced. An example for such a natural reduction is the decrease of soluble oligomannosyl residues occuring in the midcycle vaginal secretion.

We claim:

1. Method of inhibiting the transition of free HIV virus carrying an envelope glycoprotein gp120/gp160 through the cellular mucosal barrier of an organism, characterized in that said glycoprotein is blocked by increasing in the region of said mucosal barrier the concentration of a compound comprising an oligomannosyl glycan residue and/or of a compound comprising a mimic molecule of an oligomannosyl glycan residue, wherein the link of said glycoprotein to said HIV virus remains essentially unaffected.

2. Method of claim 1, characterized in that the increase of the concentration of said compound in said barrier is effected by local administration of said compound to said barrier.

3. Method of claim 1, characterized in that the increase of the concentration of said compound in said barrier is effected by stimulation of the β-andrenergic system within the said organism.

4. Method of claim 1, characterized in that the increase of the concentration of said compound in said barrier is effected by inhibition of the endogenic processing of glycans.

5. Method of any of the claims 1 to 4, characterized in that the compound is selected from the group consisting of “N-glycans, mannan, high-mannose type glycans, hybrid-type glycans, complex-type glycans, α-methylmannopyranosid, mucine, yeasts, beer yeasts, extracts of Aloe Vera, and mixtures and/or derivatives thereof”, wherein the mannose residues of said compound are essentially non-sulphated.

6. Method of claim 1 characterized in that the transition of said HIV virus through an epithalial cell barrier is inhibited.

7. A pharmaceutical composition capable of inhibiting the transition of free HIV virus carrying an envelope glycoprotein gp120/gp160 through the mucosal barrier of an organism, characterized in that it contains at least one compound comprising an oligomannosyl glycan residue and/or at least one compound comprising a mimic molecule of an oligomannosyl glycan residue for blocking said glycoprotein, wherein the link of the said glycoprotein to said HIV virus remains essentially unaffected.

8. The pharmaceutical composition of claim 7, characterized in that it is prepared for local application of said composition to an epithelial tissue of said organism.

9. A pharmaceutical composition capable of inhibiting the transition of free HIV virus carrying an envelope glycoprotein gp120/160 through the mucosal barrier of an organism, characterized in that it contains at least one inhibitor of the endogenic processing of glycans.

10. The pharmaceutical composition of claim 7, characterized in that the compound is selected from the group consisting of “N-glycans, mannan, high-mannose type glycans, hybrid-type glycans, complex-type glycans, α-methylmannopyranosid, mucine yeasts, beer yeast, extracts of Aloe vera, and mixtures and/or derivatives thereof”, wherein the mannose residues of said compound are essentially non-sulphated.

11. The pharmaceutical composition of claim 9, characterized in that it contains an inhibitor selected from the group consisting of “desoxymannojirimycin, swainsonine, desoxynojirimycin, and mixtures thereof”.

12. The pharmaceutical composition of any of the claims 7 to 11, characterized in that it further contains a pyrimidine nucleoside analogue capable of inhibiting reverse transcriptase.

13. The pharmaceutical composition of claim 12, characterized in that it further contains 3′-Azido-3′-desoxythymidin.

14. Method for preventing an infection of an organism with free HIV virus carrying an envelope glycoprotein gp120/gp160 characterized in that a compound comprising an oligomannosyl glycan residue and/or a compound comprising a mimic molecule of an oligomannosyl glycan is administered locally to epithelial tissues of said organism, wherein said glycoprotein is blocked by said compound and wherein the link of said glycoprotein to said HIV virus remains essentially unaffected.

15. Method for preventing an infection of an organism with free HIV virus carrying an envelope glycoprotein gp120/gp160, characterized in that an increase of the concentration of a compound comprising an oligomannosyl glycan residue in said barrier is effected by stimulation of the β-andrenergic system within the said organism.

16. Method for preventing an infection of an organism with free HIV virus carrying an envelope glycoprotein gp120/gp160, characterized in that an increase of the concentration of a compound comprising an oligomannosyl glycan residue in said barrier is effected by inhibition of the endogenic processing of glycans.

17. Method of claim 14, characterized in that the compound is selected from the group consisting of “N-glycans, mannan, high-mannose type glycans, hybrid-type glycans, complex-type glycans, α-methylmannopyranosid, mucine, yeasts, beer yeast, extracts of Aloe vera, and mixtures and/or derivatives thereof”, wherein the mannose residues of said compound are essentially non-sulphated.

18. Method of claim 16 characterized in that said inhibition is effected by administration of desoxymannojirimycin.

19. Method for treating an organism infected with HIV virus carrying an-envelope glycopretein gp120/gp160 characterized in that a compound comprising an oligomannosyl glycan residue and/or a compound comprising a mimic molecule of an oligomannosyl glycan residue is administered to said organism, wherein said glycoprotein is blocked by said compound and wherein the link of said glycoprotein to said HIV virus remains essentially unaffected, and that a pyrimidine nucleoside analogue capable of inhibiting reverse transcriptase is administered to said organism, wherein the administration of said compound and of said nucleoside analogue is performed simultaneously or sequentially alternating.

20. Method of claim 19, characterized in that said compound is selected from the group consisting of “N-glycans, mannan, high-mannose type glycans, hybrid-type glycans, complex-type glycans, α-methylmannopyranosid, mucine, yeasts, beer yeast, extracts of Aloe vera, and mixtures and/or derivatives thereof”, wherein the mannose residues of said compound are essentially non-sulphated, and that said nucleoside analogue is 3′-Azido-3′-desoxythymidin.
